(19) United States
(12) Patent Application Publication
(10) Pub. No.: US 2016/0068875 A1
(43) Pub. Date: Mar. 10, 2016

(54) Production of tailored PHA copolymers from natural gas

(71) Applicant: The Board of Trustees of the Leland Stanford Junior University, Palo Alto, CA (US)

(72) Inventors: Craig S. Criddle, Redwood City, CA (US); Wakuna M. Galega, Stanford, CA (US); Jaewook Myung, Stanford, CA (US)

(21) Appl. No.: 14/836,510

(22) Filed: Aug. 26, 2015

Related U.S. Application Data
(60) Provisional application No. 62/047,990, filed on Sep. 9, 2014.

(51) Int. Cl. C12P 7/62

(52) U.S. Cl. CPC C12P 7/625

(57) ABSTRACT

A method of producing polyhydroxyalkanoic acid (PHA)-producing biomass is provided that includes obtaining a methane-oxidizing inoculum, flushing the methane-oxidizing inoculum with natural gas and oxygen, amending the flushed methane-oxidizing inoculum with a fresh growth medium, using a non-aseptic bioreactor for growing a PHA-producing biomass, where the non-aseptic bioreactor is seeded with the amended methane-oxidizing inoculum, where a natural gas and oxygen mixture is added to the non-aseptic bioreactor, where a growth medium comprising ammonium and nutrients required for exponential growth is added to the non-aseptic bioreactor, harvesting a portion of the methane-oxidizing biomass and incubating the harvested portion in the absence of nitrogen and with the natural gas and oxygen mixture, where a PHA-enriched biomass is produced, purifying PHA from the PHA-enriched biomass, and adding the fresh growth medium and the natural gas and oxygen mixture to the bioreactor to re-grow the methane-oxidizing inoculum.

### CROSS-REFERENCE TO RELATED APPLICATIONS

This application claims priority from U.S. Provisional Patent Application 62/047990 filed Sep. 9, 2014, which is incorporated herein by reference.

### FIELD OF THE INVENTION

The present invention relates generally to production of polyhydroxyalkanoic acid (PHA). More particularly, the invention relates to production of PHA biopolymers from natural gas.

### BACKGROUND OF THE INVENTION

In recent years, significant research has focused on reducing fossil fuel dependence and producing polymers from renewable sources. One such approach toward sustainability is development of polyhydroxyalkanoates (PHAs) that are accumulated by many prokaryotic organisms under unbalanced growth conditions. Due to their material properties similar to those of polypropylene or polyethylene, complete biodegradability and excellent biocompatibility, these polyesters have attracted much attention. Moreover, unlike common petrochemical plastics (e.g., polyethylene, polypropylene, polystyrene), PHA-based products can be recycled without downcycling. The challenge, however, is high production cost of PHAs, which likely resulted in limited potential of commercialization. PHAs are currently commercially produced from expensive sugar feedstock, which significantly contributes to their high price.

An alternative carbon source is methane. Methane availability is not tied to the food supply, so its price is less sensitive to factors affecting the food price. Methane delivered as natural gas is especially of great interest because prices have fallen dramatically over the last decade and because the infrastructure for delivery of natural gas is ubiquitous. Shale gas is also largely methane and is increasingly accessible with new extraction technology.

Synthesis of poly-3-hydroxybutyrate (P3HB) from methane has been extensively studied. Generally, methane includes a portion of the components of natural gas, in addition to other gases that include carbon dioxide, dinitrogen, and other short alkanes.

What is needed is method of producing P3HB from natural gas.

### SUMMARY OF THE INVENTION

To address the needs in the art, a method of producing polyhydroxyalkanoic acid (PHA)-producing biomass is provided that includes obtaining a methane-oxidizing inoculum, flushing the methane-oxidizing inoculum with natural gas and oxygen, amending the flushed methane-oxidizing inoculum with a fresh growth medium, using a non-aseptic bioreactor for growing a PHA-producing biomass, where the non-aseptic bioreactor is seeded with the amended methane-oxidizing inoculum, where a natural gas and oxygen mixture is added to the non-aseptic bioreactor, where a growth medium comprising ammonium and nutrients required for exponential growth is added to the non-aseptic bioreactor, harvesting a portion of the methane-oxidizing biomass and incubating the harvested portion in the absence of nitrogen and with the natural gas and oxygen mixture, where a PHA-enriched biomass is produced, purifying PHA from the PHA-enriched biomass, and adding the fresh growth medium and the natural gas and oxygen mixture to the bioreactor to re-grow the methane-oxidizing inoculum.

According to one aspect of the invention, the grown PHA includes polymers such as P3HB, PHBV or P3HB4HB.

In another aspect of the invention, the natural gas and oxygen mixture includes a molar ratio in the range of 1:1 to 1:2.

In a further aspect of the invention, the methane-oxidizing inoculum includes activated sludge.

According to another aspect of the invention, the methane-oxidizing inoculum includes sediment.

In yet another aspect of the invention, the methane-oxidizing inoculum includes a defined mixed culture such as Type II methanotrophs, methylotrothic aerobic bacteria, or aerobic species capable of oxidizing C-2 to C-9 alkanes. In one aspect the Type II methanotrophs include species of the genera Methylocystis and Methylosinus. In another aspect, the methylotrothic aerobic bacteria, including species of the genus Hyphomicrobium. In a further aspect, the aerobic species capable of oxidizing C-2 to C-9 alkanes of the genus includes the genera Thauera, Arthrobacter, Cycloclasticus, or Colwellia.

### BRIEF DESCRIPTION OF THE DRAWINGS

FIGS. 1A-1B show (FIG. 1A) final P3HB wt % after 48-h incubation under nitrogen-limited condition, and (FIG. 1B) change of molecular weight during a 48-h nitrogen-limited P3HB accumulation step (Cycle 51), according to one embodiment of the invention.

FIG. 2 shows a flow diagram of a method of producing polyhydroxyalkanoic acid (PHA)-producing biomass, according to one embodiment of the invention.

### DETAILED DESCRIPTION

The current invention is a method of producing polyhydroxyalkanoic acid (PHA) and accumulation of poly-3-hydroxybutyrate (P3HB), from natural gas, a hydrocarbon gas mixture containing primarily methane (70-90%), but commonly including varied amounts of higher alkanes (0-20%), carbon dioxide (0-8%), nitrogen (0-5%), and hydrogen sulfide (0-5%). Microbial production of PHAs from natural gas is complicated by the nature of the gas mixture and inhibitory interactions that result when different components of the gas mixture are metabolized. For example, microorganisms that are capable of metabolizing methane (methanotrophs) are typically inhibited by the oxidation of higher alkanes in a natural gas mixture. This is due to the non-specific substrate range of methane monooxygenase, the enzyme that mediates initial attack on methane, oxidizing it to methanol. This enzyme also co-metabolizes higher alkanes, resulting in the production and accumulation of toxic alcohols. Conversely, microorganisms capable of metabolizing higher alkanes often possess monooxygenase enzymes that fortuitously cometabolize methane, resulting in the accumulation of toxic C-1 metabolites, such as methanol or formaldehyde. In this invention, defined mixed cultures and enrichments are used to prevent accumulation of toxic metabolites and enable production of PHAs under nutrient limited conditions. In one example, a methonotrophic bacterium capable of PHA production, such as Methylosinus trichosporium OB3b or Methylocystis parvus OBBP, is grown in co-culture with a second species capable of growth and PHA production upon higher alkanes, such as Thauera butanivorans, a species capable of growth upon C2-C9 alkanes. In another example, methane-oxidizing microorganisms and higher alkane-oxidizing microorganisms capable of PHA production are fed pipeline natural gas or captured natural gas that is otherwise flared or vented to the atmosphere. The resulting enrichment culture is grown under conditions favorable for the accumulation of intracellular PHA granules.

According to one embodiment, PHA production using natural gas is a two-step process. In the first step, cells are grow with natural gas in a balanced growth phase: a phase in which sufficient major and minor nutrients is present to support cell division, where in addition to organic energy and carbon sources, bacteria require a number of other nutrients including nitrogen, phosphorus, sulfur, calcium, trace metals and salts. The natural gas supports replication of both methane-oxidizing bacteria and bacteria that can oxidize higher alkanes. This is followed by an unbalanced growth phase in which one or more nutrients is limiting, preventing further cell division. Under such conditions, growth occurs through the synthesis of intracellular PHA granules, and the cells expand. During this phase, natural gas is provided to enable production of PHA co-polymers, with variable side chain composition and/or variable number of carbon atoms in the polymer backbone. The nature and percentage of co-polymer composition reflects the percentage of higher alkanes present in the natural gas. Co-polymer modifications through adjustment of the higher alkane fraction can confer many useful properties, such as impact resistance, toughness, and flexibility.

Until now, production of PHA from natural gas has not been demonstrated. The cause is inhibition due to accumulation of partial oxidation products. The current invention overcomes this limitation through the use of PHA-producing defined mixed cultures or enrichments that synergistically consume any partial oxidation products generated, thus preventing their accumulation.

During the PHA production phase, natural gas alone, or supplemented with added co-substrates, such as long chain fatty acids, are provided to enable production of customized PHA co-polymers, with variable side chain composition and/or variable number of carbon atoms in the polymer backbone.

This invention provides a methodology for convenient purification of waste PHAs and production of customized PHAs without downcycling. Companies and individuals who manufacture products with natural gas will enjoy the benefit of a cheap substrate that can be reliably and conveniently transported to fermentation facilities.

Some issues the current invention overcomes are pipeline natural gas is composed of largely methane and ethane, but the ratio of each gas component is not consistent. Further natural-gas fed enrichment was dominated by Type II methanotrophs, methylotrophs and short-chain alkane (n>1) utilizers, and use of a complex gas substrate induces fluctuations in the microbial community. Stable P3HB synthesis is enabled, and pure methane and ethane can replace natural gas without any noticeable P3HB production capacity.

According to one embodiment, a method of producing polyhydroxyalkanoic acid (PHA)-producing biomass is provided that includes obtaining a methane-oxidizing inoculum, flushing the methane-oxidizing inoculum with natural gas and oxygen, amending the flushed methane-oxidizing inoculum with a fresh growth medium, using a non-aseptic bioreactor for growing a PHA-producing biomass, where the non-aseptic bioreactor is seeded with the amended methane-oxidizing inoculum, where a natural gas and oxygen mixture is added to the non-aseptic bioreactor, where a growth medium comprising ammonium and nutrients required for exponential growth is added to the non-aseptic bioreactor, harvesting a portion of the methane-oxidizing biomass and incubating the harvested portion in the absence of nitrogen and with the natural gas and oxygen mixture, where a PHA-enriched biomass is produced, purifying PHA from the PHA-enriched biomass, and adding the fresh growth medium and the natural gas and oxygen mixture to the bioreactor to re-grow the methane-oxidizing inoculum.

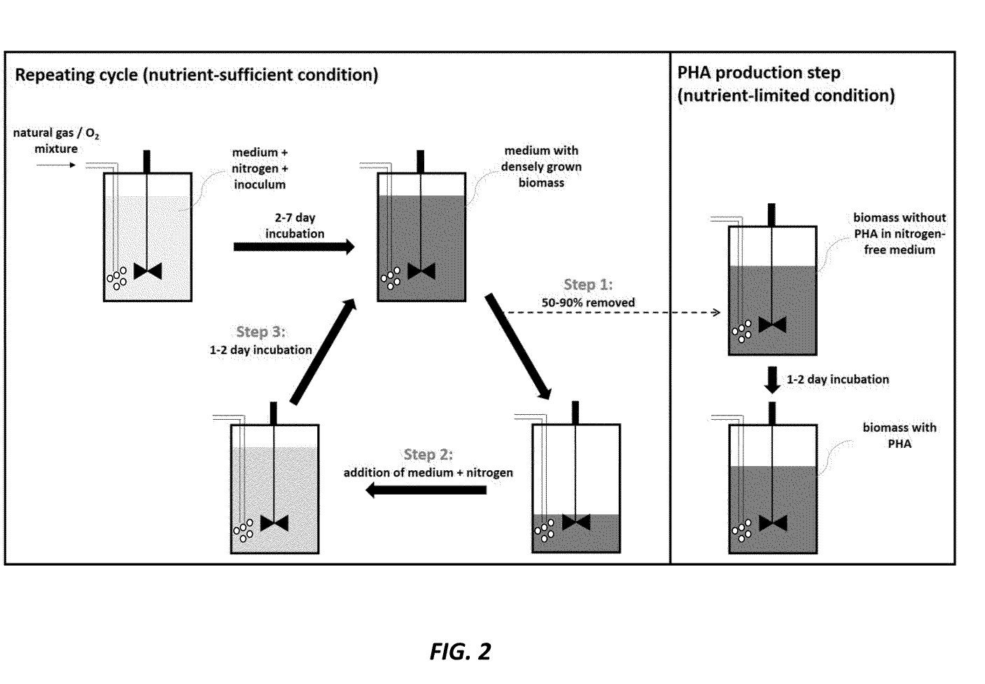

Turning now to an exemplary laboratory process, unless otherwise specified, all cultures were grown in medium JM2, where a flow diagram of the general process is shown in FIG. 2. Medium JM2 contained the following chemicals per L of solution: 2.4 mM MgSO₄.7H₂O, 0.26 mM CaCl₂, 36 mM NaHCO₃, 4.8 mM KH₂PO₄, 6.8 mM K₂HPO₄, 10.5 μM Na₂MoO₄.2H₂O, 7 μM CuSO₄.5H₂O, 200 μM Fe-EDTA, 530 μM Ca-EDTA, 5 mL trace metal solution, and 20 mL vitamin solution. The trace stock solution contained the following chemicals per L of solution: 500 mg FeSO₄.7H₂O, 400 mg ZnSO₄.7H₂O, 20 mg MnCl₂.7H₂O, 50 mg CoCl₂.6H₂O, 10 mg NiCl₂.6H₂O, 15 mg H₃BO₃ and 250 mg EDTA. The vitamin stock solution contained the following chemicals per L of solution: 2.0 mg biotin, 2.0 mg folic acid, 5.0 mg thiamine.HCl, 5.0 mg calcium pantothenate, 0.1 mg vitamin B12, 5.0 mg riboflavin and 5.0 mg nicotinamide.

All cultures were incubated in 160-mL serum bottles capped with butyl-rubber stoppers and crimp-sealed under a natural gas and oxygen (>99% purity) headspace (molar ratio 1:1.5). Liquid volume was 50 mL, and the headspace volume was 110 mL. Cultures were incubated horizontally on orbital shaker tables at 150 rpm. The incubation temperature was 30° C.

Fresh activated sludge was obtained from an aeration basin. Large material was removed by filtering through a 100-μm cell strainer. The dispersed cells were centrifuged for 15 min to create a pellet. The pellet was resuspended in medium JM2 and shaken to obtain a dispersed cell suspension. Aliquots (15 mL) of the suspension were added to two serum vials containing 35 mL of medium JM2. Every 24 h for two weeks, the headspace of each bottle was flushed with a natural gas and oxygen mixture (molar ratio of 1:1 to 1:2) and amended with 0.5 mL of ammonium stock solution (1.35 M ammonium chloride; >99.8% purity). When the culture reached a final optical density (OD₆₀₀) of 1.2, it was centrifuged (10,000×g) for 15 min, and the pellet resuspended in 15 mL of medium JM2. The suspension was divided into 5-mL aliquots for inoculation of three fed-batch serum bottle cultures. Each fed-batch culture initially contained 5 mL of inoculum, 44.5 mL of medium JM2, and 0.5 mL of ammonium stock (total volume 50 mL). After a 24-h incubation period, each of the three enrichments was subject to a long-term cyclic feeding and wasting regime, with alternating pulses of natural gas and ammonium. A repeating 48-h repeating fed-batch cycle was established enabling nearly continuous exponential growth. In Step 1, all cultures with 10 mL of carry-over culture from the previous cycle received 40 mL of fresh medium (39.5 mL of medium JM2 plus 0.5 mL of ammonium stock) and were flushed for 5 min with a natural gas and oxygen mixture (molar ratio of 1:1.5). In Step 2, all cultures were incubated at 30° C. with exponential growth over a 24-h period. In Step 3, all cultures received a second 5 min headspace flush with a natural gas and oxygen mixture (molar ratio of 1:1.5). In Step 4 all cultures were incubated at 30° C. with exponential growth over a second 24-h period. Finally, in Step 5, 40 mL of liquid was quickly removed (5 min) from all cultures, completing a cycle.

In a further aspect of the invention, the methane-oxidizing inoculum includes activated sludge.

According to another aspect of the invention, the methane-oxidizing inoculum includes sediment.

In yet another aspect of the invention, the methane-oxidizing inoculum includes a defined mixed culture such as Type II methanotrophs, methylotrothic aerobic bacteria, or aerobic species capable of oxidizing C-2 to C-9 alkanes. In one aspect the Type II methanotrophs include species of the genera Methylocystis and Methylosinus. In another aspect, the methylotrothic aerobic bacteria, including species of the genus Hyphomicrobium. In a further aspect, the aerobic species capable of oxidizing C-2 to C-9 alkanes of the genus includes the genera Thauera, Arthrobacter, Cycloclasticus, or Colwellia.

Turning now to P3HB production under nitrogen-limited growth conditions, a portion of the samples removed in Step 5 was centrifuged (10,000×g) for 15 min then suspended in fresh medium without nitrogen. The headspace of each bottle was filled with a natural gas and oxygen mixture (molar ratio of 1:1 to 1:2) at t=0 h and again at t=24 h. In some samples, natural gas was replaced with either pure methane (>99% purity), ethane (>99% purity) or propane (>99% purity) to assess P3HB production using different alkanes. After 48 h of incubation, cells were harvested from the triplicate samples by centrifugation (10,000×g) and freeze-dried. Preserved samples were assayed for P3HB content.

After establishing a repeating cycle of operation, 100-μL samples were removed from each enrichment culture, and the genomic DNA (gDNA) extracted using the FastDNA SPIN Kit for Soil, as per the manufacture's protocol. Bacterial 16S rRNA was amplified using the bacterial primers BAC-8F (5′-AGAGTTTGATCCTGGCTCAG-3′) and BAC-1492R (5′-CGGCTACCTTGTTACGACTT-3′). A Polymerase chain reaction (PCR) was performed using Accuprime Taq DNA Polymerase System and the following thermocycling steps: (i) 94° C. for 5 min; (ii) 30 cycles consisting of 94° C. for 30 s, 55° C. for 30 s, 68° C. for 80 s; and (iii) an extension at 68° C. for 10 min. Amplicon presence and quality of PCR reaction were verified via 1.5% agarose gel electrophoresis.

PCR products were purified using QIAquick PCR Purification Kit, then cloned using pGEM-T Easy Vector System with JM109 competent Escherichia coli cells per the manufacture's protocol. Randomly selected clones were sequenced, generating 120 near-full length 16S rRNA gene sequences. Retrieved DNA sequences were compared with reference sequences using Basic Local Alignment Search Tool (BLAST).

To analyze concentrations of methane, ethane, propane, oxygen, nitrogen and carbon dioxide, 0.5 mL of gas phase from natural gas pipeline and each enrichment culture was injected onto GOW-MAC gas chromatograph with an Altech CTR 1 column and a thermal conductivity detector. The following method parameters were used: injector, 120° C.; column, 60° C.; detector, 120° C.; and current, 150 mV. Peak areas of each gas were compared to gas standards (>99% purity) and quantified using software.

To analyze total suspended solids (TSS), 0.5-5.0 mL of cell suspension was filtered through pre-washed, dried, and pre-weighted 0.2-μm membrane filters. The filtered cells and membrane filters were dried at 105° C. for 24 h, then weighed on an AD-6 autobalance.

Between 5 and 10 mg of freeze-dried biomass were weighed then transferred to a 12-mL glass vial. Each vial was amended with 2 mL of methanol containing sulfuric acid (3%, vol/vol) and benzoic acid (0.25 mg/mL methanol), supplemented with 2 mL of chloroform, and sealed with a Teflon-lined plastic cap. All vials were shaken then heated at 95-100° C. for 3.5 h. After cooling to room temperature, 1 mL of deionized water was added to create an aqueous phase separated from the chloroform organic phase. The reaction cocktail was mixed on a vortex mixer for 30 s then allowed to partition until phase separation was complete. The organic phase was sampled by syringe and analyzed using a GC equipped with a column containing 5% phenyl-methylpolysiloxane and a flame ionization detector. DL-hydroxybutyric acid sodium salt was used to prepare external calibration curves. The P3HB content (wt %, w_{P3HB}/w_{CDW}) of the samples was calculated by normalizing to initial dry mass.

P3HB granules were extracted from the cells by suspending 500 mg of freeze-dried cell material in 50-mL Milli-Q water, adding 400 mg of sodium dodecyl sulfate (>99.0% purity) and 360 mg of EDTA, followed by heating to 60° C. for 60 min to induce cell lysis. The solution was centrifuged (10,000μg) for 15 min, and the pellet washed three times with deionized water. To purify the P3HB, pellets were washed with a 50-mL sodium hypochlorite (bleach) solution (Clorox 6.15%), incubated at 30° C. with continuous stirring for 60 min, and centrifuged (10,000 μg) for 15 min. Sample pellets were then washed three times with deionized water.

Molecular weights of P3HB were evaluated using gel permeation chromatography (GPC). Sample pellets dissolved in chloroform at a concentration of 5 mg/mL for 90 min at 60° C. were filtered through a 0.2-μm PTFE filter, then analyzed with an ultra fast liquid chromatography system equipped with a RID-10A refraction index detector. The GPC was equipped with a divinylbenzene (DVG) gel column (500 Å) and DVB gel analytical columns (10⁵ Å). The temperature of the columns was maintained at 40° C., and the flow rate of the mobile phase (chloroform) was 1 mL min⁻¹. Molecular weights were calibrated with polystyrene standards.

Carbon balances were prepared for reactants and products for each serum bottle culture (in C-mmol). Biomass was assumed to have an empirical composition of C₅H₇O₂N. the electron balances were also calculated for each serum bottle culture (in e-mmol) for reactant and products relative to the reference oxidation states of carbon dioxide and water.

Turning now to the pipeline natural gas composition,

Table 1 illustrates the composition of natural gas used in this study. Methane was the largest component (>95%), and ethane was the second-largest component (μ2.8%) of the natural gas. Propane and butane were present in trace amount. While the order of abundance among different gas components did not change, standard deviations of each gas component throughout the test period was not negligible.

TABLE 1
Composition of pipeline natural gas used in this study. Triplicate natural
gas samples were analyzed at nine random time points
(Cycle 1, 4, 5, 7, 10, 22, 35, 44 and 47) throughout the test period.

| | | Carbon | | | | Bu- |
|---|---|---|---|---|---|---|
| | Nitrogen | dioxide | Methane | Ethane | Propane | tane |
| Mole | 0.4 ± 0.2 | 1.2 ± 0.5 | 95.5 ± 2.5 | 2.8 ± 0.5 | 0.1 ± 0.1 | <0.1 |
| % | | | | | | |

Patterns of substrate consumption and community composition were evaluated for the serum bottle enrichment cultures fed natural gas on a repeating cycle, which illustrates the pattern of a typical 48-h repeating cycle (Cycle 52). The errors bars represent standard deviations for triplicate batch cultures.

After 10 cycles of operation, Methylocystis dominated the natural gas-fed enrichment (see Table 2), Hyphomicrobium was the second-largest genus in the community. Rhodococcus, Nocardia and other minor genera including Burkholderia, Rhodopseudomonas and Castellaniella accounted for the remaining bacteria. The order of abundant genera remained stable, but the ratio of each genus fluctuated throughout the test period (Cycle 10 to Cycle 44).

TABLE 2
Most probable affiliation of the genus-level bacterial community
structures based on 16S rRNA gene sequences retrieved from the
triplicate natural gas-utilizing microbial enrichment cultures.

| | Proportions (%) | | |
|---|---|---|---|
| Affiliation | Cycle 10 | Cycle 22 | Cycle 44 |
| Proteobacteria | | | |
| Alphaproteobacteria | | | |
| Rhizobiales | | | |
| Methylocystis | 55.8 ± 3.5 | 79.0 ± 4.2 | 72.8 ± 3.8 |
| Hyphomicrobium | 10.2 ± 1.8 | 7.9 ± 0.4 | 12.8 ± 0.9 |
| Rhodopseudomonas | 0.5 ± 0.1 | 0.4 ± 0.1 | 0.5 ± 0.1 |
| Betaproteobacteria | | | |
| Burkholderiales | | | |
| Burkholderia | 1.2 ± 0.1 | 0.4 ± 0.1 | 0.6 ± 0.1 |
| Castellaniella | 0.5 ± 0.1 | 0.2 ± 0.1 | 0.6 ± 0.1 |
| Actinobacteria | | | |
| Actinomycetales | | | |
| Rhodococcus | 2.3 ± 0.2 | 2.5 ± 0.1 | 3.9 ± 0.3 |
| Nocardia | 1.9 ± 0.1 | 1.0 ± 0.2 | 2.2 ± 0.2 |
| Others | 27.6 ± 3.0 | 8.6 ± 0.5 | 7.6 ± 0.6 |

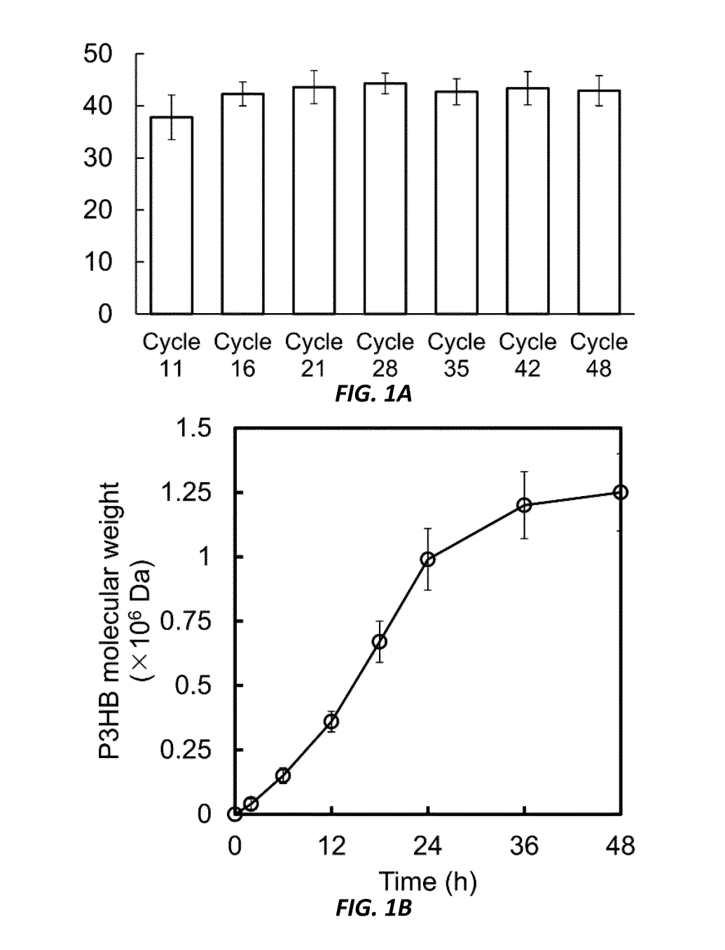

Regarding fed-batch enrichments and the nitrogen-limited P3HB production step, natural gas-fed enrichment cultures produced P3HB when incubated under nitrogen-limited condition. FIG. 1A shows the wt % of resulting P3HB granules from Cycle 11 to Cycle 48. The wt % of resulting P3HB granules was steady at >40% after Cycle 16. FIG. 1B shows the change of molecular during a 48-h P3HB production step. At t=24 h, the molecular weight exceeds 10⁶ Da, and remains steady ˜1.2×10⁶ Da after t=36 h. It also indicates that the natural gas-fed enrichment is capable of producing high molecular weight P3HB.

In some cycles, natural gas was replaced with synthetic short-chain alkane mixtures.

Table 3 illustrates the final P3HB wt % and the specific P3HB accumulation rate when the enrichment was fed with these mixtures. When methane, ethane or their mixtures were used, we did not observe any significant difference compared to the control. However, use of propane decreased the final PHA wt % from ˜40 to ˜30 wt % and also decreased the specific P3HB accumulation rate by ˜66%.

TABLE 3
Patterns of P3HB accumulation when natural gas was replaced with
synthetic short-chain alkane mixtures.

| | | | | Specific P3HB |
|---|---|---|---|---|
| Ratio of each gas in mixture | | | Final | accumulation rate |
| Methane | Ethane | Propane | P3HB | (mg P3HB mg |
| (%) | (%) | (%) | wt % | TSS⁻¹ h⁻¹') |
| 100 | 0 | 0 | 40 ± 3 | 0.011 ± 0.002 |
| 0 | 100 | 0 | 40 ± 4 | 0.011 ± 0.002 |
| 0 | 0 | 100 | 31 ± 2 | 0.004 ± 0.001 |
| 50 | 50 | 0 | 41 ± 4 | 0.012 ± 0.002 |

| | | |
|---|---|---|
| Control (natural gas) | 42 ± 3 | 0.012 ± 0.002 |

To assess process stoichiometry, we computed carbon and electron balances for both the 48-h repeating cycle (

Table 4) and the 48-h nitrogen-limited step ( Table 5). All triplicate measurements had relative errors<13%. During the 48-h repeating cycle, short-chain alkanes (methane, ethane and propane) were used as carbon and electron sources to produce non-P3HB biomass. NH₄⁺ was the primary nitrogen source for assimilation. During the 48-h nitrogen-limited step, the short-chain alkanes were primarily used to produce P3HB biomass.

TABLE 4
Cell growth during a 48-h repeating cycle (Cycle 21): mole balances
for carbon and electron.

| | Reactant moles | | | Product moles | |
|---|---|---|---|---|---|
| Re- | Carbon | Electron | | Carbon | Electron |
| actants | (C-mmol) | (e-mmol) | Products | (C-mmol) | (e-mmol) |
| Meth- | 3.6 ± 0.4 | 29 ± 3 | Non- | 3.3 ± 0.4 | 13 ± 2 |
| ane | | | P3HB | | |
| | | | biomass | | |
| | | | (C₅H₇O₂N) | | |
| Ethane | 0.20 ± | 1.4 ± 0.14 | Carbon | 0.81 ± | 0 |
| | 0.02 | | dioxide | 0.060 | |
| Pro- | 0.013 ± | 0.089 ± | Water | 0 | 20 ± 2 |
| pane | 0.003 | 0.021 | | | |
| Oxygen | 0 | 0 | | | |
| SUM | 3.8 ± 0.4 | 30 ± 3 | SUM | 4.1 ± 0.4 | 33 ± 2 |

TABLE 5
P3HB accumulation during a 48-h nitrogen-limited step (Cycle 23):
mole balances for carbon and electron.

| | Reactant moles | | | Product moles | |
|---|---|---|---|---|---|
| Re- | Carbon | Electron | | Carbon | Electron |
| actants | (C-mmol) | (e-mmol) | Products | (C-mmol) | (e-mmol) |
| Meth- | 3.6 ± 0.4 | 29 ± 3 | Non- | 0.038 ± | 0.15 ± |
| ane | | | P3HB | 0.003 | 0.01 |
| | | | biomass | | |
| | | | (C₅H₇O₂N) | | |
| Ethane | 0.20 ± | 1.4 ± 0.1 | P3HB | 3.0 ± 0.4 | 11 ± 1 |
| | 0.01 | | biomass | | |
| | | | (C₄H₆O₂) | | |
| Pro- | 0.016 ± | 0.11 ± | Carbon | 0.91 ± | 0 |
| pane | 0.002 | 0.01 | dioxide | 0.07 | |
| Oxygen | 0 | 0 | Water | 0 | 18 ± 2 |
| SUM | 3.8 ± 0.4 | 30 ± 3 | SUM | 4.0 ± 0.4 | 29 ± 2 |

Some previous studies reported P3HB production using natural gas. However, it should be noted that the composition of natural varies greatly depending on the source and suppliers. While gas from the same source has a similar composition, it is not totally stable as indicated in

Table 1. This complexity creates uncertainty in maintaining stable methanotrophic community as well as producing stable biopolymer products. In this respect, this study provides very important insight on direct use of complex mixture natural gas on bioreactors.

Metabolic specialization is a general biological principle that shapes the assembly of microbial communities. Availability of complex carbon sources mainly consisting of short-chain alkanes developed quite interesting microbial community illustrated in

Table 2. Methylocystis is a P3HB-producing Type II methane-utilizers, and Hyphomicrobium is facultative methylotrophic genus that can grow on C2 and C2 compounds and accumulate intracellular PHA. Rhodococcus and Nocardia are short alkane degraders, and are known PHA producers.

Generally, it is known that high-purity methane should be supplied to methanotrophic cultures since low-purity methane or natural gas contains methanotrophic inhibitors such as acetylene, which will prevent methanotrophic growth even at very low concentrations.

For the methanotrophic biotechnology platform using natural gas, its use as a feedstock for methanotrophs could enable economies of scale through large-scale centralized production and, at the same time, create opportunities for distributed production at small scale. Methanotrophic biotechnology could provide a platform for synthesis of PHA and other products from natural gas and other sources of methane.

Over time, natural gas could be replaced by purified and compressed methane produced by anaerobic digestion of organic residues. Natural gas could thus become a bridge to renewable production of PHAs and other high value products.

Natural gas can be a cost-effective feedstock for P3HB production, and could potentially displace cultivated feedstock.

Methanotrophic enrichment produces PHBV and P3HB4HB copolymers when amended with odd carbon fatty acids along with methane. However, natural gas enrichment fed odd carbon alkane (propane) as a sole carbon source produced P3HB homopolymer. This implies that metabolic pathways for incorporation of alkane and fatty acids should differ in natural gas enrichments.

However, some microorganisms synthesize only P3HB even when cultivated on the sources with an odd number of carbon atoms.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. All such variations are considered to be within the scope and spirit of the present invention as defined by the following claims and their legal equivalents.

What is claimed:

1. A method of producing polyhydroxyalkanoic acid (PHA)-producing biomass, comprising:
a. obtaining a methane-oxidizing inoculum;
b. flushing said methane-oxidizing inoculum with natural gas and oxygen;
c. amending said flushed methane-oxidizing inoculum with a fresh growth medium;
d. using a non-aseptic bioreactor for growing a PHA-producing biomass, wherein said non-aseptic bioreactor is seeded with said amended methane-oxidizing inoculum, wherein a natural gas and oxygen mixture is added to said non-aseptic bioreactor, wherein a growth medium comprising ammonium and nutrients required for exponential growth is added to said non-aseptic bioreactor;
e. harvesting a portion of said methane-oxidizing biomass and incubating said harvested portion in the absence of nitrogen and with said natural gas and oxygen mixture, wherein a PHA-enriched biomass is produced;
f. purifying PHA from said PHA-enriched biomass; and
g. adding said fresh growth medium and said natural gas and oxygen mixture to said non-aseptic bioreactor to re-grow said methane-oxidizing inoculum.

2. The method of producing polyhydroxyalkanoic acid (PHA)-producing biomass according to claim 1, wherein said grown PHA comprises polymers selected from the group consisting of P3HB, PHBV and P3HB4HB.

3. The method of producing polyhydroxyalkanoic acid (PHA)-producing biomass according to claim 1, wherein said natural gas and oxygen mixture comprises a molar ratio in the range of 1:1 to 1:2.

4. The method of producing polyhydroxyalkanoic acid (PHA)-producing biomass according to claim 1, wherein said methane-oxidizing inoculum comprises activated sludge.

5. The method of producing polyhydroxyalkanoic acid (PHA)-producing biomass according to claim 1, wherein said methane-oxidizing inoculum comprises sediment.

6. The method of producing polyhydroxyalkanoic acid (PHA)-producing biomass according to claim 1, wherein said methane-oxidizing inoculum comprises a defined mixed culture selected from the group consisting of Type II methanotrophs, methylotrothic aerobic bacteria, and aerobic species capable of oxidizing C-2 to C-9 alkanes.

7. The method of producing polyhydroxyalkanoic acid (PHA)-producing biomass according to claim 6, wherein said Type II methanotrophs comprise species of the genera Methylocystis and Methylosinus.

8. The method of producing polyhydroxyalkanoic acid (PHA)-producing biomass according to claim 6, wherein said methylotrothic aerobic bacteria, including species of the genus Hyphomicrobium.

9. The method of producing polyhydroxyalkanoic acid (PHA)-producing biomass according to claim 6, wherein said aerobic species capable of oxidizing C-2 to C-9 alkanes of the genus comprise species selected from the group consisting of the genera Thauera, Arthrobacter, Cycloclasticus, and Colwellia.
